(19) United States
(12) Patent Application Publication
(10) Pub. No.: US 2026/0049011 A1
(43) Pub. Date: Feb. 19, 2026

(54) COMPOSITIONS AND METHODS FOR CONTROLLING SLUDGE BED PROPERTIES AND IMPROVING LIME SOFTENING PROCESS EFFICIENCY

(71) Applicant: ChampionX LLC, Sugar Land, TX (US)

(72) Inventors: Ming-Zhao JIN, Sherwood Park (CA); Dingzheng YANG, Sherwood Park (CA); Song GAO, Sherwood Park (CA)

(73) Assignee: ChampionX LLC, Sugar Land, TX (US)

(21) Appl. No.: 19/290,556

(22) Filed: Aug. 5, 2025

Related U.S. Application Data
(60) Provisional application No. 63/684,193, filed on Aug. 16, 2024.

(51) Int. Cl. C02F 1/52; C02F 1/00; C02F 5/06; G01N 33/38

(52) U.S. Cl. CPC C02F 1/5209; C02F 1/004; C02F 1/5272; C02F 1/5281; G01N 33/38; C02F 2001/007; C02F 5/06

(57) ABSTRACT

The present disclosure relates to compositions including coagulants, testing methods and devices, and methods used in connection with lime softening processes, such as warm or hot lime softening processes. The compositions, devices, and methods disclosed herein may be used to improve sludge bed properties in lime softening processes, such as warm lime softening process or hot lime softening processes.

### TECHNICAL FIELD

The present disclosure generally relates to compositions, devices, and methods used in connection with lime softening processes, such as warm or hot lime softening processes. More particularly, the disclosure provides devices, compositions comprising coagulants and/or flocculants, methods of using the coagulants and/or flocculants, and a testing methodology aimed at improving sludge bed properties in lime softening processes.

### BACKGROUND

The application of warm lime softeners is crucial in various industrial processes, such as power plants, refineries, and Steam Assisted Gravity Drainage (SAGD) steam generation for enhanced oil recovery. The warm lime softening process removes hardness and silica from water, preventing scale formation in boilers and heat exchangers, which is essential for maintaining efficiency and longevity of the water treatment system. The efficiency and effectiveness of warm lime softening operations are significantly influenced by the flowability and characteristics of the precipitated solids bed, or sludge bed. Proper flowability ensures that precipitated calcium carbonate and magnesium hydroxide can be easily settled and removed, thereby preventing clogging and ensuring smooth operation.

Additionally, the sludge bed acts as a filter, further improving water quality by trapping fine particles and enhancing the clarity of the treated water. The particle size, density, and electrokinetic properties of the sludge impact sedimentation rates and sludge handling. Optimal sludge bed management, including maintaining appropriate height and density, is critical for maximizing the contact time for chemical reactions and ensuring effective removal of impurities.

The properties of the sludge bed in warm lime softeners are most influenced by coagulants and flocculants because these chemicals determine how particles coalesce and aggregate to form the sludge bed. However, finding and adjusting new coagulants and flocculants to improve sludge bed properties in industrial warm lime softening systems is a significant challenge. This is because it takes weeks to months for a new chemical to generate a sludge bed with new properties in an industrial setting. While laboratory tests, such as jar tests, can be used to assess how new coagulants and flocculants reduce water turbidity, they fall short in predicting the long-term sludge bed properties that these chemicals will produce in full-scale operations. This discrepancy arises because jar tests primarily evaluate immediate particle aggregation and turbidity reduction, but do not replicate the dynamic and complex conditions of an industrial sludge bed formation. Industrial trials have found that some chemicals, which perform well in lab tests, can generate very solid (low flowability) sludge beds that hinder the smooth rotation of rakes and sludge recycling. Such solid beds also fail, or are less effective, in serving as filters for hardness and silica removal, highlighting the importance of field trials in addition to lab tests to accurately predict the performance of new coagulants and flocculants in WLS systems.

Currently, no chemistry or method is guaranteed to consistently generate qualified sludge bed properties across different warm lime softening applications and varying feedwater conditions. Therefore, there is a need for an improved coagulant/flocculant formulation and a reliable method that can replicate the desired sludge bed properties of incumbent products, while also improving water treatment outcomes by reducing dosage, improving turbidity, and enhancing hardness and silica removal.

### BRIEF SUMMARY

The present disclosure provides compositions, testing methods and devices, and methods used in connection with lime softening processes, such as warm or hot lime softening processes.

In some embodiments, a method of the present disclosure includes adding calcium hydroxide and optionally magnesium hydroxide, magnesium oxide, sodium hydroxide, and/or sodium carbonate to a feed water, transporting the feed water to a first channel, adding the composition to the feed water in the first channel, transporting the feed water in the first channel to a first settling tank, wherein the feed water passes through a first sludge bed in the first settling tank, withdrawing a first sample of the feed water from the first settling tank downstream of the first sludge bed, and measuring a property of the first sample.

In some embodiments, a device of the present disclosure comprises a chemical feed tank in fluid communication via a conduit with an inlet of a mixing tank, the mixing tank further comprising an outlet in fluid communication with a feed water conduit, the feed water conduit comprising a first conduit leading to an inlet of a first mixing device, and a first coagulant feed tank in fluid communication with the first conduit via a third conduit, wherein an outlet of the first mixing device is in fluid communication via a fifth conduit with an inlet of a first settling tank, the first settling tank comprising a first sludge bed.

In some embodiments, a method of improving a lime softening process is disclosed herein. The method comprises adding a composition to a lime softening process feed water upstream of a settling tank, the settling tank comprising a sludge bed, transporting the feed water through the sludge bed of the settling tank, and forming a precipitated solid within the feed water, wherein the composition comprises a first coagulant and a second coagulant, wherein the first and second coagulants are independently selected from the group consisting of aluminum sulfate, ferric chloride, ferric sulfate, polyaluminum chloride, a polyamine, polydiallyldimethylammonium chloride, dimethylamine-epichlorohydrin-ethylene diamine, epichlorohydrin-dimethylamine, acrylamide-dimethylaminoethyl acrylate methyl chloride quaternary, zinc sulfate, titanium sulfate, tannins, chitosan, starch, a starch derivative, polyepichlorohydrin-dimethylamine, polyferric sulfate, aluminum chlorohydrate, and any combination thereof.

In some embodiments, a composition of the present disclosure includes a first coagulant and a second coagulant, wherein the first and second coagulants are independently selected from the group consisting of aluminum sulfate, ferric chloride, ferric sulfate, polyaluminum chloride, a polyamine, polydiallyldimethylammonium chloride, dimethylamine-epichlorohydrin-ethylene diamine, epichlorohydrin-dimethylamine, acrylamide-dimethylaminoethyl acrylate methyl chloride quaternary, zinc sulfate, titanium sulfate, tannins, chitosan, starch, a starch derivative, polyepichlorohydrin-dimethylamine, polyferric sulfate, aluminum chlorohydrate, and any combination thereof.

The foregoing has outlined rather broadly the features and technical advantages of the present disclosure in order that the detailed description that follows may be better understood. Additional features and advantages of the disclosure will be described hereinafter that form the subject of the claims of this application.

### BRIEF DESCRIPTION OF THE SEVERAL VIEWS OF THE DRAWINGS

A detailed description of the invention is hereafter described with specific reference being made to the drawings in which:

FIG. 1 shows an example of a testing device in accordance with the present disclosure.

### DETAILED DESCRIPTION

Various embodiments are described below with reference to the drawings in which like elements generally are referred to by like numerals. The relationship and functioning of the various elements of the embodiments may better be understood by reference to the following detailed description. However, elements and embodiments are not strictly limited to those illustrated in the drawings or described below.

Examples of methods and materials are described below, although methods and materials similar or equivalent to those described herein can be used in practice or testing of the present disclosure. All publications, patent applications, patents and other reference materials mentioned herein are incorporated by reference in their entirety. The materials, methods, and examples disclosed herein are illustrative only and not intended to be limiting.

Unless otherwise defined, all technical and scientific terms used herein have the same meaning as commonly understood by one of ordinary skill in the art. In case of conflict, the present document, including definitions, will control.

Compounds of the present disclosure may be substituted with suitable substituents. The term “suitable substituent,” as used herein, is intended to mean a chemically acceptable functional group, preferably a moiety that does not negate the activity of the compounds. Such suitable substituents include, but are not limited to, halo groups, perfluoroalkyl groups, perfluoroalkoxy groups, alkyl groups, alkenyl groups, alkynyl groups, hydroxy groups, oxo groups, mercapto groups, alkylthio groups, alkoxy groups, aryl or heteroaryl groups, aryloxy or heteroaryloxy groups, aralkyl or heteroaralkyl groups, aralkoxy or heteroaralkoxy groups, HO—(C═O)-groups, heterocylic groups, cycloalkyl groups, amino groups, alkyl and dialkylamino groups, carbamoyl groups, alkylcarbonyl groups, alkoxycarbonyl groups, alkylaminocarbonyl groups, dialkylamino carbonyl groups, arylcarbonyl groups, aryloxycarbonyl groups, alkylsulfonyl groups, and arylsulfonyl groups. In some embodiments, suitable substituents may include halogen, an unsubstituted C₁-C₁₂ alkyl group, an unsubstituted C₄-C₆ aryl group, or an unsubstituted C₁-C₁₀ alkoxy group. Those skilled in the art will appreciate that many substituents can be substituted by additional substituents.

The term “substituted” as in “substituted alkyl,” means that in the group in question (e.g., the alkyl group), at least one hydrogen atom bound to a carbon atom is replaced with one or more substituent groups, such as hydroxy (—OH), alkylthio, phosphino, amido (—CON(R_{A})(R_{B}), wherein R_{A} and R_{B} are independently hydrogen, alkyl, or aryl), amino(—N(R_{A})(R_{B}), wherein R_{A} and R_{B} are independently hydrogen, alkyl, or aryl), halo (fluoro, chloro, bromo, or iodo), silyl, nitro (—NO₂), an ether (—OR_{A} wherein R_{A} is alkyl or aryl), an ester (—OC(O)R_{A} wherein R_{A} is alkyl or aryl), keto (—C(O)R_{A} wherein R_{A} is alkyl or aryl), heterocyclo, and the like.

When the term “substituted” introduces a list of possible substituted groups, it is intended that the term apply to every member of that group. That is, the phrase “optionally substituted alkyl or aryl” is to be interpreted as “optionally substituted alkyl or optionally substituted aryl. ”

The terms “polymer,” “copolymer,” “polymerize,” “copolymerize,” and the like include not only polymers comprising two monomer residues and polymerization of two different monomers together, but also include (co)polymers comprising more than two monomer residues and polymerizing together more than two or more other monomers. For example, a polymer as disclosed herein includes a terpolymer, a tetrapolymer, polymers comprising more than four different monomers, as well as polymers comprising, consisting of, or consisting essentially of two different monomer residues. Additionally, a “polymer” as disclosed herein may also include a homopolymer, which is a polymer comprising a single type of monomer unit.

Unless specified differently, the polymers of the present disclosure may be linear, branched, crosslinked, structured, synthetic, semi-synthetic, natural, and/or functionally modified. A polymer of the present disclosure can be in the form of a solution, a dry powder, a liquid, or a dispersion, for example.

The term “turbidity” as used herein refers to a measurement of water clarity. Turbidity is measured according to the amount of light that passes through a sample, where a clear sample has low turbidity and a murkier sample has higher turbidity. The more the light is scattered or blocked during testing, the higher the turbidity according to the unit of measurement Nephelometric Turbidity Unit (NTU).

The present disclosure provides devices, compositions, and methods used in connection with lime softening processes, such as warm or hot lime softening processes.

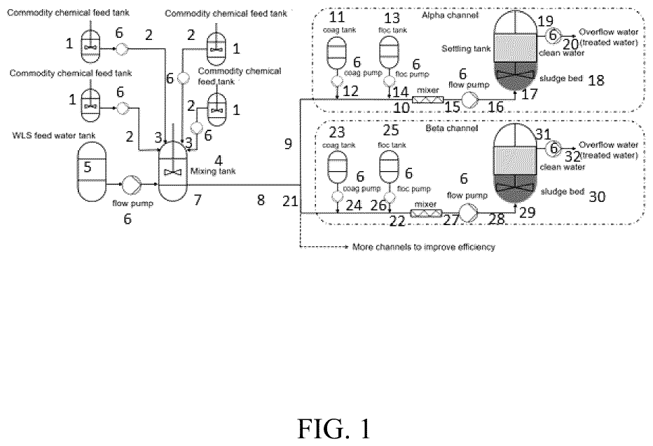

As can be seen in FIG. 1, a device of the present disclosure may include one or more chemical feed tanks (1) in fluid communication via a conduit (2) with an inlet (3) of a mixing tank (4). While FIG. 1 shows a device having four chemical feed tanks, the device may have any number of feed tanks, such as 1-10, 1-8, 1-6, 1-4, 1-2, 1, 2, 3, 4, 5, 6, 7, 8, 9, 10, or more. Each chemical feed tank may comprise a single chemical or a mixture of chemicals, such as calcium hydroxide, magnesium hydroxide, magnesium oxide, sodium hydroxide, sodium carbonate, or any combination thereof, optionally diluted in a solvent, such as water.

Each conduit (2) may comprise a flow pump (6) to assist with transporting the chemical to the mixing tank (4). The device may also include a feed water tank (5), which transports feed water through a pump (6) and into the mixing tank (4). The mixing tank (4) further comprises an outlet (7) in fluid communication with a feed water conduit (8), the feed water conduit comprising a first conduit (9) leading to an inlet (10) of a first mixing device.

A device as disclosed herein may also comprise a first coagulant feed tank (11) in fluid communication with the first conduit (9) via a third conduit (12). The third conduit (12) may also comprise a pump (6). A device as disclosed herein may optionally comprise a first flocculant feed tank (13) in fluid communication with the first conduit (9) via a seventh conduit (14). The seventh conduit (14) may also comprise a pump (6).

An outlet (15) of the first mixing device is in fluid communication via a fifth conduit (16) with an inlet (17) of a first settling tank, the first settling tank comprising a first sludge bed (18). The first sludge bed (18) is disposed at the bottom of the first settling tank and the first settling tank also includes an outlet (19) where treated water may exit the settling tank via a ninth conduit (20).

Although the ninth conduit (20) is shown at a particular location in FIG. 1, it can be located at any portion of the settling tank above the sludge bed.

While the device has thus far been disclosed as having a first conduit (9) leading to at least a first coagulant feed tank (11), a first mixing device, and a first settling tank, the device may include any number of additional conduits, such as 1, 2, 3, 4, 5, 6, 7, 8, 9, 10, or more additional conduits, each additional conduit leading to at least an additional coagulant feed tank, mixing device, and settling tank, although the additional conduit may optionally lead to an additional flocculant feed tank.

For example, FIG. 1 depicts a device wherein the feed water conduit (8) comprises a second conduit (21) leading to an inlet (22) of a second mixing device. A second coagulant feed tank (23) is included and is in fluid communication with the second conduit (21) via a fourth conduit (24). The device may optionally comprise a second flocculant feed tank (25) in fluid communication with the second conduit (21) via an eighth conduit (26). The eighth conduit (26) may also comprise a pump (6). An outlet (27) of the second mixing device is in fluid communication via a sixth conduit (28) with an inlet (29) of a second settling tank. A second sludge bed (30) is disposed at the bottom of the second settling tank and the second settling tank also includes an outlet (31) where treated water may exit the settling tank via a tenth conduit (32). Although the tenth conduit (32) is shown at a particular location in FIG. 1, it can be located at any portion of the settling tank above the sludge bed.

Again, depending on the number of channels splitting off from the feed water conduit (8), the device may comprise a plurality of coagulant feed tanks, a plurality of flocculant feed tanks, a plurality of mixing devices, and a plurality of settling tanks. Typically, each channel will include a conduit, a coagulant feed tank in fluid communication with the conduit, a mixing device in fluid communication with the conduit, and a mixing tank in fluid communication with the conduit. Each channel may optionally comprise a flocculant feed tank and any conduit in the device or channel may optionally include a flow pump.

The devices disclosed herein may be used in methods for evaluating compositions. For example, a method may include adding calcium hydroxide, magnesium hydroxide, magnesium oxide, sodium hydroxide, sodium carbonate, or any combination thereof to a feed water. In certain embodiments, the chemicals may be added individually or in any combination to a mixing tank and after an appropriate amount of mixing (a few seconds, a few minutes, a few hours, etc.) the mixture may exit the tank and flow into a conduit comprising feed water leading to a first channel.

The mixture will then pass through a mixing device and into a bottom portion of a settling tank, where it will then pass through a sludge bed and into a mid-portion of the settling tank. After being treated by the sludge bed, the clean/treated water may flow through a conduit out of an upper mid-portion of the settling tank.

The temperature of the feed water is not particularly limited when conducting methods of the present disclosure. In an illustrative embodiment, the temperature is from about 40° C. to about 90° C., such as about 50° C. to about 80° C., about 50° C. to about 70° C., or about 50° C. to about 60° C. In other embodiments, the temperature is from about 100° C. to about 130° C., such as about 110° C. to about 120° C.

A composition comprising a coagulant or a mixture of coagulants may be added to the feed water in the first channel and transported to a first settling tank, wherein the feed water passes through a first sludge bed in the first settling tank. A first sample of the feed water from the first settling tank may be withdrawn from the settling tank downstream of the first sludge bed, such as through the conduit at the upper mid-portion of the settling tank. Then, a property of the first sample may be measured.

A variety of water properties may be measured, such as hardness (calcium and/or magnesium ion concentration), turbidity, and/or silica concentration. One of ordinary skill in the art understands how to measure these properties and what devices may be used to measure the properties. For example, turbidity meters useful for measuring water turbidity are known in the art and, for example, a Hach DR6000 (or other series) device may be used to determine silica concentration, calcium concentration, and/or magnesium concentration.

As described above, a device disclosed herein may include any number of channels and the disclosed testing methods may therefore include the additional steps of transporting the feed water to a second channel, adding a second composition comprising one or more coagulants to the feed water in the second channel, and transporting the mixture to a second settling tank, wherein the feed water passes through a second sludge bed in the second settling tank. A second sample of the feed water from the second settling tank may be withdrawn from the settling tank downstream of the second sludge bed, such as through the conduit at the upper mid-portion of the settling tank. Then, a property of the second sample may be measured.

In certain embodiments, one or more flocculants may optionally be added to the feed water in each channel, mixed with the coagulant(s) in the mixing device, and transported to the settling tank. In some embodiments, the same flocculant or flocculant combination is added to the feed water of each channel.

Typically, a property, such as hardness, turbidity, and/or silica concentration of the first sample is measured and the same property (or combination of properties) of the second sample is measured. Then, the measured properties are compared in order to evaluate the effectiveness of the first and/or second composition.

For example, the first composition may comprise a first coagulant and a second coagulant and the second composition may comprise a third coagulant. Upon comparing the measured property (or properties) of the water, one can determine whether the first composition is more effective or whether the second composition is more effective.

For instance, a first composition may comprise a first coagulant and a second coagulant. A second composition may comprise a third coagulant. If it is determined that the feed water of the second settling tank is less turbid, has lower silica levels, and/or has lower levels of hardness, a weight ratio of the first coagulant and the second coagulant in the first composition may be adjusted in an attempt to make the first composition more effective at reducing turbidity, hardness, and/or silica levels in the feed water.

In some embodiments, the first and second coagulants are different. The third coagulant may be different from the first and second coagulants or it may be the same as the first or second coagulant. The more channels the device has, the more coagulants (or coagulant combinations) can be simultaneously tested.

In some embodiments, each coagulant (or coagulant combination) is kept isolated within its respective channel. For example, if a device includes two channels, the composition added to the feed water of the first channel is not added to the feed water in the second channel and the second composition, which is added to the feed water of the second channel, is not added to the feed water in the first channel.

The methods disclosed herein may also include the steps of removing a portion of the first sludge bed and adding it to a first container, such as a graduated cylinder or the like. The portion of the sludge bed may be allowed to settle in the container and the settling rate may be calculated.

A portion of a different sludge bed, such as the second sludge bed, may be removed from a different settling tank, such as the second settling tank, and it may be added to a different container, such as the second container. The portion of the second sludge bed may be allowed to settle in the container and the settling rate may be calculated. The settling rate of the first sludge bed may be compared to the settling rate of the second sludge bed, third sludge bed, fourth sludge bed, etc., depending upon how many different sludge beds are included in the device.

In certain embodiments, the amount of coagulant in the first composition and/or the second composition introduced into one of the feed water channels, such as channel A, can be adjusted to achieve a settling rate in channel A that is substantially identical to, or within a desired range relative to, the settling rate in a second channel, such as channel B. For example, if the settling rate in channel B, using its specific coagulant composition in a 500 ml cylinder, is 3.0 ml/min during the first hour, the ratio of two coagulants in channel A may be adjusted to achieve a settling rate between 2.5 and 3.0 ml/min, resulting in a denser bed. Alternatively, the ratio of coagulants in channel A can be modified to reach a settling rate between 3.0 and 4.0 ml/min, producing a looser bed. Typically, the coagulant used in channel B is the standard (incumbent) in lime softener, and its performance (resulting in either an over-loose or over-dense bed for industrial lime softener) provides a reference settling range to optimize the formulation of the composition in channel A to the desired range.

In certain testing methods disclosed herein, the device processes about 10 to about 100 liters of feed water to generate an approximately 200 ml to about 2,000 ml sludge bed, allowing continuous assessment of both sludge and water properties. While more sludge can be generated, a sludge bed volume between 200 ml and 2,000 ml is sufficient for settling tests used to optimize the coagulant formulation. The generated sludge bed may be observed during the testing process and may undergo settling tests to characterize its flowability and settling rate. The methods disclosed herein allow for precise adjustment of coagulant formulations to achieve the desired sludge bed properties based on current incumbent product operations, ensuring consistent performance across various warm lime softening applications and feedwater conditions. The coagulant formulations developed through the testing methods disclosed herein can significantly improve the efficiency and effectiveness of the warm lime softening process.

The devices and testing methods disclosed herein allow for the evaluation of sludge bed properties for incumbent and new coagulant formulations. The methods of the present disclosure aim to achieve the same or substantially similar sludge bed properties as the incumbent product by adjusting the ratio of two or more coagulant components in a composition through continuous unit testing to identify the optimized formulation for sludge properties generation. Performing this type of analysis in an industrial setting could take weeks or even months for new sludge to build up and be ready for analysis. It would also require shutting down the operation in order to access the sludge, which is highly risky and could jeopardize the entire operation. However, the presently disclosed device and methods allow for testing sludge beds in as little as a few minutes to a few hours (e.g., 5 minutes, 30 minutes, 60 minutes, 120 minutes, etc.). The inventive devices and procedures are designed to optimize multiple component formulations of coagulants and optionally flocculants to control sludge bed properties and improve the efficiency of warm and/or hot lime softening processes.

Any composition of the present disclosure may include one or more coagulants. Illustrative, non-limiting examples of coagulants include aluminum sulfate, ferric chloride, ferric sulfate, polyaluminum chloride, a polyamine, polydiallyldimethylammonium chloride, dimethylamine-epichlorohydrin-ethylene diamine, epichlorohydrin-dimethylamine, acrylamide-dimethylaminoethyl acrylate methyl chloride quaternary, zinc sulfate, titanium sulfate, tannins, chitosan, starch, a starch derivative, polyepichlorohydrin-dimethylamine, polyferric sulfate, aluminum chlorohydrate, and any combination thereof.

In some embodiments, a composition of the present disclosure includes two different coagulants. In these embodiments, the composition may include any weight ratio of the first coagulant to the second coagulant. For example, a composition of the present disclosure may comprise a weight ratio of the first coagulant to the second coagulant of about 1:100 to about 100:1, such as about 1:75, about 1:50, about 1:25, about 1:15, about 1:10, about 1:5, about 1:2, about 1:1, about 2:1, about 5:1, about 10:1, about 15:1, about 25:1, about 50:1, or about 75:1.

In an illustrative embodiment, a composition of the present disclosure includes a first coagulant comprising dimethylamine-epichlorohydrin-ethylene diamine and a second coagulant comprising polyepichlorohydrin-dimethylamine.

Compositions of the present disclosure may optionally include a flocculant. Illustrative, non-limiting examples of flocculants include polyacrylamide, a polyethyleneimine, starch, a starch derivative, chitosan, magnafloc, a polyacrylate, a poly-triethylamine quat, and any combination thereof.

Any composition disclosed herein may also include a solvent, such as water. The amount of solvent is not particularly limited. In some embodiments, a composition comprises from about 1 wt. % to about 99 wt. % of the solvent, such as about 10 wt. % to about 99 wt. %, about 20 wt. % to about 99 wt. %, about 30 wt. % to about 99 wt. %, about 40 wt. % to about 99 wt. %, about 50 wt. % to about 99 wt. %, about 60 wt. % to about 99 wt. %, about 70 wt. % to about 99 wt. %, about 80 wt. % to about 99 wt. %, or about 90 wt. % to about 99 wt. %.

In certain embodiments, compositions of the present disclosure may exclude diallyldimethylammonium chloride. In certain embodiments, compositions of the present disclosure exclude a cationic polymer.

In some embodiments, the compositions of the present disclosure include at least a first coagulant and a second coagulant. By combining two or more coagulants, a composition of the present disclosure can match the desired sludge properties of an incumbent product, enhance turbidity reduction, lower chemical dosage, and improve hardness and silica removal efficiency.

The compositions of the present disclosure may be used in connection with methods for improving a lime softening process, such as a warm or hot lime softening process. In some embodiments, the compositions comprising one or more coagulants may be added to a lime softening process feed water upstream of a settling tank, which comprises a sludge bed. Optionally, one or more flocculants may be added as well. The coagulants promote precipitation of feed water contaminants, such as hardness and/or silica, and the feed water will pass through the sludge bed, which may act to filter materials, such as precipitated solids, and subsequently exit the settling tank with reduced hardness, silica levels, etc.

The amount of coagulant (or combined coagulant) added to the feed water is not particularly limited. In some embodiments, from about 1 ppm to about 1,000 ppm of each coagulant of a composition may be added, such as from about 1 ppm to about 800 ppm, about 1 ppm to about 600 ppm, about 1 ppm to about 400 ppm, about 1 ppm to about 200 ppm, about 1 ppm to about 100 ppm, about 1 ppm to about 50 ppm, or about 1 ppm to about 25 ppm. The foregoing amounts also apply to any flocculant that may be added in a method of the present disclosure.

In certain embodiments, methods of the present disclosure may exclude a step of adding diallyldimethylammonium chloride to the feed water. In certain embodiments, methods of the present disclosure may exclude a step of adding a cationic polymer to the feed water.

The foregoing may be better understood by reference to the following examples, which are intended for illustrative purposes and are not intended to limit the scope of the disclosure or its application in any way.

### EXAMPLES

An embodiment of the presently disclosed device, substantially similar to the device of FIG. 1, was used to evaluate and optimize various compositions of the present disclosure. The device included a small-scale, multi-channel continuous unit. The unit processed about 20 to about 60 liters of warm lime softening feed water and generated about a 1 liter sludge bed, allowing continuous assessment of both sludge and water properties. Magnesium hydroxide, lime, and soda ash were added to the feed water. Also, various coagulant compositions were added, such as a composition comprising an epichlorohydrin dimethyl amine copolymer and, in some cases, a flocculant, such as a polyacrylate, was separately added. The generated sludge bed was observed during the testing process and underwent settling tests to characterize its flowability and settling rate. Another test included comparing the settling rate of the generated sludge in a graduated cylinder. In some cases, multiple test rounds were conducted to allow for adjustment of the formulation in an attempt to match incumbent sludge properties.

Water properties that were measured included turbidity, hardness, and silica concentration using a turbidity meter and a Hach DR6000 for the silica and water hardness measurements. Sludge settling tests were conducted in graduated cylinders.

In one test, a composition comprising a dimethylamine-epichlorohydrin-ethylene diamine polymer and polyepichlorohydrin-dimethylamine was used in a sludge settling test. Compositions comprising weight ratios of 8:2, 5:5 and 2:8 dimethylamine-epichlorohydrin-ethylene diamine polymer to polyepichlorohydrin-dimethylamine were tested. The composition comprising the weight ratio of 2:8 was selected because it matched the settling rate of the incumbent coagulant, which included an epichlorohydrin dimethyl amine copolymer. Additionally, this composition showed dosage reduction and turbidity improvement of treated water over the incumbent coagulant.

All of the compositions and methods disclosed and claimed herein can be made and executed without undue experimentation in light of the present disclosure. While this invention may be embodied in many different forms, there are described in detail herein specific preferred embodiments of the invention.

The present disclosure is an exemplification of the principles of the invention and is not intended to limit the invention to the particular embodiments illustrated. In addition, unless expressly stated to the contrary, use of the term “a” is intended to include “at least one” or “one or more. ” For example, “a coagulant” is intended to include “at least one coagulant” or “one or more coagulants. ”

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges (including all fractional and whole values) subsumed therein.

Any composition disclosed herein may comprise, consist of, or consist essentially of any element, component and/or ingredient disclosed herein or any combination of two or more of the elements, components or ingredients disclosed herein.

Any method disclosed herein may comprise, consist of, or consist essentially of any method step disclosed herein or any combination of two or more of the method steps disclosed herein.

The transitional phrase “comprising,” which is synonymous with “including,” “containing,” or “characterized by,” is inclusive or open-ended and does not exclude additional, un-recited elements, components, ingredients and/or method steps.

The transitional phrase “consisting of” excludes any element, component, ingredient, and/or method step not specified in the claim.

The transitional phrase “consisting essentially of” limits the scope of a claim to the specified elements, components, ingredients and/or steps, as well as those that do not materially affect the basic and novel characteristic(s) of the claimed invention.

Unless specified otherwise, all molecular weights referred to herein are weight average molecular weights and all viscosities were measured at 25° C. with neat (not diluted) polymers.

As used herein, the term “about” refers to the cited value being within the errors arising from the standard deviation found in their respective testing measurements, and if those errors cannot be determined, then “about” may refer to, for example, within 5%, 4%, 3%, 2%, or 1% of the cited value.

Furthermore, the invention encompasses any and all possible combinations of some or all of the various embodiments described herein. It should also be understood that various changes and modifications to the presently preferred embodiments described herein will be apparent to those skilled in the art. Such changes and modifications can be made without departing from the spirit and scope of the invention and without diminishing its intended advantages. It is therefore intended that such changes and modifications be covered by the appended claims.

What is claimed is:

1. A method of evaluating a composition, comprising:
adding calcium hydroxide and optionally magnesium hydroxide, magnesium oxide, sodium hydroxide, and/or sodium carbonate to a feed water,
transporting the feed water to a first channel,
adding the composition to the feed water in the first channel,
transporting the feed water in the first channel to a first settling tank, wherein the feed water passes through a first sludge bed in the first settling tank,
withdrawing a first sample of the feed water from the first settling tank downstream of the first sludge bed, and
measuring a property of the first sample.

2. The method of claim 1, further comprising:
transporting the feed water to a second channel,
adding a second composition to the feed water in the second channel,
transporting the feed water in the second channel to a second settling tank, wherein the feed water passes through a second sludge bed in the second settling tank,
withdrawing a second sample of the feed water from the second settling tank downstream of the second sludge bed,
measuring a property of the second sample, and
comparing the property of the first sample with the property of the second sample.

3. The method of claim 2, wherein the composition comprises a first coagulant and the second composition comprises a second coagulant.

4. The method of claim 1, wherein the property is selected from hardness, turbidity, and/or silica concentration.

5. The method of claim 2, further comprising removing a portion of the first sludge bed, adding it to a first container, allowing it to settle, determining a first settling rate, removing a portion of the second sludge bed, adding it to a second container, allowing it to settle, determining a second settling rate, and comparing the first settling rate to the second settling rate.

6. The method of claim 5, further comprising adjusting an amount of the first coagulant being added to the feed water in the first channel until the first settling rate is within a desired range based on industrial lime softener requirements.

7. The method of claim 3, wherein the first and second coagulants are independently selected from the group consisting of aluminum sulfate, ferric chloride, ferric sulfate, polyaluminum chloride, a polyamine, polydiallyldimethylammonium chloride, dimethylamine-epichlorohydrin-ethylene diamine, epichlorohydrin-dimethylamine, acrylamide-dimethylaminoethyl acrylate methyl chloride quaternary, zinc sulfate, titanium sulfate, tannins, chitosan, starch, a starch derivative, polyepichlorohydrin-dimethylamine, polyferric sulfate, aluminum chlorohydrate, diallyldimethylammonium chloride, and any combination thereof.

8. The method of claim 3, wherein the first and second coagulants are different.

9. The method of claim 3, wherein the first coagulant comprises a mixture of dimethylamine-epichlorohydrin-ethylene diamine and polyepichlorohydrin-dimethylamine.

10. The method of claim 1, further comprising adding a flocculant to the feed water in the first channel and/or adding a flocculant to the feed water in the second channel, wherein the flocculant is selected from the group consisting of a polyacrylamide, a polyethyleneimine, starch, a starch derivative, chitosan, magnafloc, a polyacrylate, a poly-triethylamine quat, and any combination thereof.

11. The method of claim 2, wherein the composition is not added to the feed water in the second channel and the second composition is not added to the feed water in the first channel.

12. A device, comprising:
a chemical feed tank in fluid communication via a conduit with an inlet of a mixing tank, the mixing tank further comprising an outlet in fluid communication with a feed water conduit, the feed water conduit comprising a first conduit leading to an inlet of a first mixing device, and
a first coagulant feed tank in fluid communication with the first conduit via a third conduit,
wherein an outlet of the first mixing device is in fluid communication via a fifth conduit with an inlet of a first settling tank, the first settling tank comprising a first sludge bed.

13. The device of claim 12, wherein the feed water conduit comprises a second conduit leading to an inlet of a second mixing device, and a second coagulant feed tank in fluid communication with the second conduit via a fourth conduit, wherein an outlet of the second mixing device is in fluid communication via a sixth conduit with an inlet of a second settling tank, the second settling tank comprising a second sludge bed.

14. The device of claim 12, further comprising a plurality of chemical feed tanks in fluid communication with the inlet of the mixing tank, a first flocculant feed tank in fluid communication with the first conduit via a seventh conduit, and/or a second flocculant feed tank in fluid communication with the second conduit via an eighth conduit.

15. A method of improving a lime softening process, comprising:
adding a composition to a lime softening process feed water upstream of a settling tank, the settling tank comprising a sludge bed,
transporting the feed water through the sludge bed of the settling tank, and
forming a precipitated solid within the feed water, wherein the composition comprises a first coagulant and a second coagulant, wherein the first and second coagulants are independently selected from the group consisting of aluminum sulfate, ferric chloride, ferric sulfate, polyaluminum chloride, a polyamine, polydiallyldimethylammonium chloride, dimethylamine-epichlorohydrin-ethylene diamine, epichlorohydrin-dimethylamine, acrylamide-dimethylaminoethyl acrylate methyl chloride quaternary, zinc sulfate, titanium sulfate, tannins, chitosan, starch, a starch derivative, polyepichlorohydrin-dimethylamine, polyferric sulfate, aluminum chlorohydrate, and any combination thereof.

16. The method of claim 15, further comprising adding a flocculant to the settling tank.

17. The method of claim 15, further comprising filtering the precipitated solid to form a treated water.

18. The method of claim 15, wherein the composition comprises a weight ratio of the first coagulant to the second coagulant of about 1:100 to about 100:1.

19. The method of claim 15, wherein the first coagulant comprises dimethylamine-epichlorohydrin-ethylene diamine and the second coagulant comprises polyepichlorohydrin-dimethylamine.

20. The method of claim 15, wherein the method excludes adding diallyldimethylammonium chloride to the feed water and/or wherein the method excludes adding a cationic polymer to the feed water.
